(19) United States
(12) Patent Application Publication
(10) Pub. No.: US 2007/0143873 A1
(43) Pub. Date: Jun. 21, 2007

(54) Potassium channels

(76) Inventors: Rejane Pratelli, Bozel (FR); Eric Hosy, Miramas (FR); Benoit Lacombe, Montpellier (FR); Charles Romieu, Jacou (FR); Laurent Torregrosa, Luc/Orbieu (FR); Jean-Baptiste Thibaud, Montpellier (FR); Herve Sentenac, Montpellier (FR)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 10/588,361

(22) PCT Filed: Jan. 31, 2005
(86) PCT No.: PCT/FR05/50060

§ 371 Date: Oct. 24, 2006

(30) Foreign Application Priority Data
Feb. 3, 2004 (FR) 0450199

(51) Int. Cl. G01N 33/53; C07H 21/04; A01H 1/00; C12N 15/82

(52) U.S. Cl. 800/278; 435/7.1; 530/370; 530/388.1; 435/419; 435/468; 536/23.6

(57) ABSTRACT

The invention relates to the use of a gene encoding a new outward rectifier potassium channel of Vitis vinifera to modify a phenotype relating to a size and the organic acid composition of grape berries. This gene is used in such a manner that it increases the size of such berries and the quantity of tartaric acid accumulated in them. To achieve this provision is made, for example, for the product of the gene to be over-expressed by transgenesis. The invention also relates to a marker comprising a nucleotidic sequence encoding a polypeptidic sequence having at least a 40% similarity with a polypeptidic sequence deduced from the VvSOR gene (SEQ ID No. 1). This marker enables genes in other species of plants to be identified, these genes encoding new potassium channels similar to the potassium channel of grape berries, so that the size and/or organic acid composition of storage organs of plants can also be modified.

### TECHNICAL FIELD

This invention relates to an improvement in the agronomic qualities of a plant in order to obtain improved characteristics for industry, in particular for the farm-produce industries, for example the production of grape berries of different sizes having improved organic acid contents (including tartaric acid). More precisely, the object of this invention is to modify a size of the storage organs such as fruits and to improve the quantity, content or composition of organic acids in these organs by the same method.

### STATE OF THE ART

1) Size of the Storage Organs

A cultivated plant generally has a stem and at least one storage organ. Stem is understood to mean an axial section of the plant which projects above the soil, grows in the opposite direction to the roots and bears leaves and the storage organ. Storage organ refers to any organ likely to be consumed by a living being, e.g. a grain, a fleshy or oleaginous fruit, but also vegetables or tubers such as potatoes. The storage organ is temporary linked to at least one of the ends of the stem. Therefore the sap produced travels from the stem to the storage organs.

Plant species are known which naturally form storage organs having “small sized” or “large sized” phenotypes relative to a reference storage organ size of the same species. The reference size of a storage organ is understood to mean an average size of storage organs calculated from a given sample of “wild” storage organs of the same species.

For reasons of convenience of production, transformation or consumption, it may be interesting to modify the size of the storage organs in order to increase or reduce their volume. For example, by increasing the volume of the storage organs to obtain storage organs that have a large sized phenotype, it is possible to avoid increasing the number of stems of plants to be cultivated in a field. Or conversely, by reducing the volume of storage organs to obtain storage organs having a small sized phenotype, it is possible, for example, to meet the expectations of the consumers who want to eat such miniaturised storage organs in just a few mouthfuls. In particular, it is known that large volume tomatoes can be obtained which are particularly suited for the culinary preparation of stuffed tomatoes. Alternatively, a method is also known for obtaining small sized tomatoes or tomatoes less commonly referred to as “cherry tomatoes” normally eaten as an aperitif.

2) Composition of Organic Acids

The increase in mass associated with an increase in the size of a vegetable organ generally involves the accumulation of a larger quantity of water and mineral and organic elements (and the opposite is the case for a reduction in size).

Among the mineral elements whose level of accumulation is likely to be modified in parallel with the mass of a vegetable organ, mention may be made of potassium, which quite generally is the mineral ion found in most abundance in living cells.

The soluble organic elements which are accumulated in the cells accompanying potassium include, quite generally, the organic acids. In fact, the organic acids are the principal chemical species contributing the neutralisation of the positive charge applied by the potassium accumulated in the cells.

Therefore the adjustment of the level of accumulation of potassium in a vegetable organ is likely to be accompanied not only by a variation in the mass of this organ, but also the accumulation of the organic acids in the same organ.

The organic acids produced by the cellular metabolism determine the acidity of the cellular content. This acidity, which varies considerably from one type of cell to another, from one tissue to another and from one species to another, is often a major organoleptic, even technological factor, the control of which is sought as soon as a particular part of a plant is destined for animal or human consumption, whether in the raw state, for example the search for fruits of varying acidity, or after any transformation process, in the case of vines, for example, all the processes employed being aimed at obtaining wine from grape berries.

The principal organic acid accumulated in the cells of plants is often malic acid, but other organic acids may be accumulated in substantial quantities, according to the species of plant or organ.

In the case of the grape berry, another major organic acid is tartaric acid. The accumulation of tartaric acid is a relatively rare phenomenon in the vegetable kingdom, and in this regard the vine is one of the remarkable exceptions as a species of major agro-economic interest. Tartaric acid is subject to very little degradation in living cells, which means that the quantity synthesised in a given stage of development remains accumulated for a long time in the cells.

Thus in the course of development of the grape berry tartaric acid and malic acid are the two principal organic acids accumulated in the cells, and consequently the two main contributors to acidity of the must, then the vine obtained after the grape harvest. After the alcoholic fermentation produced by the yeasts, the malic acid present in the must is subjected to bacterial fermentation and is partly degraded, to a greater or lesser extent, in lactic acid. This malo-lactic fermentation, which converts a double acid to a single acid, the role of tartaric acid in determining the acidity of the wine is therefore an essential one.

This combination of facts, well known among oenologists, indicates that a high tartrate/malate ratio in the berries at the time of harvesting is an extremely positive, and therefore sought after factor.

Technical Problem

At the present time it is difficult to influence the size of the storage organs. For example, in order to increase the size of the storage organs to obtain a better yield, fertilisers are generally used. Such fertilisers are specially selected to meet the nutritional requirements of the plant and to compensate for any nutritional deficiencies of the soil. For example, fertilisers supply elements essential for the production of proteins and cellular constituents for the plant.

However, the fertiliser may be a product that is harmful to the environment. The addition of fertiliser gives rise, in particular, to pollution of the soil and ground water. The discharge of nitrates into the soil poses environmental problems that are difficult to resolve. Moreover, it is necessary to add fertiliser regularly because the nutritive elements contained in this fertiliser are exhausted during consumption by the plant.

Furthermore, in order to reduce the size of the storage organs, cross-generations of plants may be necessary in order to select the desired size. Finally, it may be the case that the “small size” or “large size” phenotype is unstable from one generation to another.

Moreover, a modification of the size need not be expressed in a degradation in the organoleptic quality of the storage organ, for example the acidity of the fruit.

It is therefore desirable to find solutions that enable the organoleptic and/or nutritional quality parameters to be maintained or improved whilst modifying the size of the storage organ.

The invention solves this problem by making use of a method that is able to act on both these parameters, independently or simultaneously. For this purpose the method according to the invention is based on controlling the mechanisms of accumulation of potassium in the organ.

In fact, as explained above, a modification of the potassium content of a storage organ is likely to influence both the growth of the said organ, and hence its ultimate size, and the content and relative composition of organic acids in the said organ.

The object of this invention is therefore to make use of the same processes, the modification not only of the size of a storage organ of a plant but also the modification of the quantity of organic acids accumulated in this organ. It is possible to modify solely the size of a storage organ or the quantity of organic acids. It is also possible to modify in parallel the size of a storage organ and the quantity of organic acids accumulated in the said storage organ.

There is a wide variety of organic acids present in the cells of a plant, and this invention may therefore have as its application a modification not only of the total quantity of organic acids accumulated in the organ concerned, but also of the distribution of the total organic acidity between the different types of organic acids. The methods according to this invention are able to modify significantly the quantity of organic acids accumulated in a storage organ of a plant. In particular, the methods according to this invention may enable the ratio of the quantity of accumulated tartaric acid in the storage organs to the quantity of accumulated malic acid in the said storage organs to be modified. According to exemplary embodiments of the methods according to the invention, the quantity of tartaric acid may be increased or decreased.

One of the objects of the invention is therefore to be able to influence the size and/or composition of organic acids in the storage organs, according to the requirements of the user, without the disadvantages associated, for example, with fertiliser additions referred to above.

Solutions Provided by the Invention

The inventors discovered that the during the development of the berry of the vine Vitis vinifera the potassium was substantially and constantly accumulated in the exocarp and in the cells of the vascular tissue binding the pedicel to the berry. Conversely, the inventors observed that during this same development the concentration of potassium in the cells of the berry tends to decrease.

Such observations led the inventors to concern themselves more specifically to identifying a system for the transmembranous transfer of potassium ions in the plant Vitis Vinifera. From genes already identified in Arabidopsis encoding outward rectifier potassium channels of the Shaker type, the inventors were able to identify a VvSOR gene in Vitis Vinifera which encodes a new outward rectifier potassium channel similar to a outward rectifier potassium channel already isolated in Arabidopsis.

In a number of experiments the inventors discovered that by over-expressing this gene in the vine, the size of the berries was increased. On the other hand, the over-expression of this gene may also cause a substantial increase in the quantity of an organic acid, such as tartaric acid, produced and accumulated in the berry. Therefore the VvSOR gene encodes a potassium out-truck involved in the growth of the grape berries and determining the accumulation of tartaric acid in the berry, which constitutes an element of the organoleptic and nutritional quality of the berry, particularly relating to the transformation of the berry to wine and the resultant organoleptic qualities of the wine.

Through this VvSOR a further object of the invention is to detect in a large number of plant species a similar gene encoding a outward rectifier potassium channel. This similar gene may then be used both to modulate the growth of the storage organs of each of the species for which the gene of interest has been isolated. This similar gene may also be used to modify the contents of certain organic acids in thee organs, independently or simultaneously.

### SUMMARY OF THE INVENTION

The principle object of the invention is therefore a method for obtaining a plant transformed on the basis of a phenotype relative to the size of a storage organ of the plant or the organic acid composition of this organ, characterised in that it comprises the following stage:
the modification, in the cells of the storage organ or in the tissues supplying the storage organ, the expression of a gene encoding a outward rectifier potassium channel.

The invention therefore also relates to a methods for selecting a plant on the basis of a phenotype relating to the size of a storage organ of the plant or the organic acid composition of that organ, characterised in that the expression of a gene encoding a outward rectifier potassium channel in the cells of the storage organs or in the tissues supplying the storage organs is measured.

The invention relates to a cell of a plant, characterised in that it over-expresses a gene encoding a outward rectifier potassium channel whose polypeptidic sequence has at least a 40% similarity with a polypeptidic sequence deduced from the nucleotidic sequence SEQ ID No. 1.

The invention also relates to a plant, characterised in that it over-expresses a gene encoding a outward rectifier potassium channel whose polypeptidic sequence has at least a 40% similarity with a polypeptidic sequence deduced from the sequence SEQ ID No. 1.

A further object of the invention is the use of a gene encoding a outward rectifier potassium channel to modify in a plant a phenotype relating to the size of at least one storage organ or the organic acid composition of that organ.

A further object of the invention is an antibody, characterised in that it is directed against all or part of a polypeptide resulting from the expression a gene encoding a outward rectifier potassium channel.

The object of the invention is a method for detecting the presence of all or part of a polypeptide resulting from the expression of a gene encoding a outward rectifier potassium channel in a sample containing a mixture of polypeptides, characterised in that it comprises the following stages:
putting the sample in contact with the antibody previously described, and
detecting an antigen/antibody complex formed.

And finally, a further object of the invention is a kit for detecting all or part of a polypeptidic sequence resulting from a gene encoding a outward rectifier potassium channel, characterised in that it comprises the antibody previously described.

### DETAILED DESCRIPTION

One of the objects of the invention is to enable a plant whose phenotype, relating to the size of a storage organ and/or the organic acid composition of that organ, is modified, to be reliably obtained. To achieve this the object of the invention is a method one of whose stages consists in modifying, in the cells of the storage organ or the tissues supplying it, an expression of a gene encoding a outward rectifier potassium channel.

As previously mentioned a plant is defined as a vegetable organism comprising a steam and at least one storage organ, the stem being intended temporarily to support this storage organ. Stem is understood to mean a an axial section of the plant which projects above the soil, grows in the opposite direction to the roots and bears leaves and the storage organs. Storage organ is defined as any organ capable of being consumed by a living being, such as a fleshy fruit, an oleaginous fruit, seeds, but also vegetables or tubers such as potatoes, for example. The storage organ is temporarily linked to at least one of the ends of the stem. Therefore the sap produced passes from the stem to the storage organs.

“Gene” is understood to refer to an ordered sequence of nucleotides which occupies a precise position on a particular chromosome and which constitutes genetic information whose transmission is hereditary. The genes correspond to a portion of a DNA molecule. They have the capacity to replicate. Genes represent elementary physical and functional units of heredity. All the genes of an organism constitute its genome.

The terms “nucleic acids”, “polynucleotides”, “oligonucleotides” or even “nucleotidic sequences” incorporate the RNA, DNA, DNAc sequences or even hybrid RNA/DNA sequences of more than one nucleotide, whether in the form of a single strand or duplex strand.

The term “nucleotide” designates both natural nucleotides (A, T, G, C) and modified nucleotides which comprise at least one modification, such as an analogue of a purine, an analogue of a pyrimidine or an analogue sugar.

For the purposes of this invention a first polynucleotide is considered to be “complementary” to a second polynucleotide when each base of the first nucleotide is matched to the complementary base of the second polynucleotide whose orientation is reversed. The complementary bases are A and T (or A and U) and C and G.

The modification of the expression of a gene in the vegetable cell may be carried out by vegetable transgenesis. Transgenesis is a transformation of the vegetable cell, which may be carried out by methods known to the person skilled in the art. More particularly, transgenesis is understood to be a number of operations that enable transgenic organisms to be obtained, i.e. organisms whose genetic inheritance (genotype) has been modified. Genetic inheritance is understood to mean all the nucleotidic sequences contained in at least one cell of a living organism. The inheritance can be modified by introducing into the cell an expression cassette obtained by genetic engineering and comprising an exogenic or foreign gene. Expression cassette is understood to mean a nucleotidic sequence encoding at least start and end transcription signals. Exogenic gene is understood to refer to a gene deriving from genetic manipulation whether aimed or not at modifying one gene in particular in the cell. In the invention this particular gene is the sequence SEQ ID No. 1 or a sequence encoding a polypeptidic sequence comprising a similarity of at least 40% with a polypeptidic sequence deduced from the sequence SEQ ID No.1. When introduced into this expression cassette, the exogenic gene will be transcribed in messenger RNA in the cell thanks to the actual signals of the cassette.

The foreign gene may be introduced directly into the cell by direct micro-injection of this foreign gene into to plant embryoids, by infiltration in a vacuum, by electroporation, by direct precipitation by means of polyethylene glycol (PEG) or by gun bombardment of particles covered with the plasmidic DNA of interest.

The foreign DNA may also be transferred by infecting the plant with a bacterial colony, in particular Agrobacterium.

Numerous methods of prior art may easily be used by the person skilled in the art to obtain plants or cells transformed by a gene encoding a potassium channel according to the invention.

These DNA transfer methods are aimed either at activating an expression of a gene in the cell (or cellular gene), or deactivating this same expression. The cellular gene may be activated either by inserting several copies of the same gene (or foreign gene) in a cellular genome or by placing the encoding sequence of the transgene under the control of a so-called strong promoter (i.e. resulting in a high level of expression), or by inserting a single copy of the gene and a power transcription factor of the gene. The deactivation of the cellular gene may be carried out, for example, by inserting a foreign gene whose RNAm is complementary to the RNAm of the gene of interest. The two RNAm's of cellular and foreign origin are therefore hybridised and no protein can be produced.

According to a particular embodiment of the invention, the method for obtaining plants whose phenotype relating to the size and/or the organic acid composition of the storage organs is modified can be implemented by transforming at least one cell of the plant of interest with a gene encoding a outward rectifier potassium channel. The transformed cells are then selected and a transformed plant is regenerated from transformed cells. The cell to be transformed may be selected from cells of a plant able to reproduce an entire organism.

According to a particular embodiment of the invention the gene whose expression is modified encodes a polypeptidic sequence having a similarity of at least 40% with a polypeptidic sequence deduced from the nucleotidic sequence SEQ ID No.1.

A polypeptidic sequence is understood to mean a chain of amino acids. The percentage of similarity between two sequences of nucleotides or amino acids, within the meaning of this invention, may be determined by comparing two sequences optimally aligned through a window of comparison.

The part of the nucleotidic or polypeptidic sequence in the window of comparison may therefore include substitutions in relation to the reference sequence in order to obtain an optimum alignment of the two sequences.

The percentage similarity is calculated between two polypeptidic sequences. This percentage similarity is calculated by determining the number of positions to which two amino acid residues correspond in an alignment whilst belonging to the same physico-chemical family of amino acids, then by dividing this number by the total number of positions in the window of comparison, and finally by multiplying the result by one hundred to obtain the percentage similarity.

The optimum alignment of the sequences for the comparison may be carried out by data processing on the basis of known algorithms.

The percentage similarity of sequences is preferably determined by means the BLAST software (BLAST version 2.06 of September 1998), using the default parameters only.

The SEQ ID No. 1 sequence corresponds to a DNAc which encodes a new outward rectifier potassium channel of Vitis Vinifera and which has been detected by means of two genes encoding similar potassium channels in Arabidopsis thaliana.

Below 40% similarity of polypeptidic sequence, the polypeptidic sequence identified in a given plant species risks not corresponding to a gene encoding a potassium channel similar to the outward rectifier potassium channel encoded by the SEQ ID No.2 gene. Such a sampled sequence may not correspond to a gene likely to be involved in the modification of the phenotype relating to the size or the organic acid composition of storage organs. It is therefore possible to obtain in each of the given species of plants a gene encoding a polypeptidic sequence having at least a 40% similarity with a polypeptidic sequence deduced from the gene of the SEQ ID No.1, encoding a outward rectifier potassium channel in Vitis Vinifera. For example, a nucleotidic sequence encoding a polypeptidic sequence having at least a 40$ similarity with a polypeptidic sequence deduced from the sequence SEQ ID No. 1 in potatoes or in rice may be identified, and such a gene may be used to modify the phenotype relating to the size or organic acid composition of storage organs of these two plants or other plant species.

According to a preferred embodiment of the invention it has been decided to modify the expression of the gene of the outward rectifier potassium channel in order to increase the size of the storage organ. However, it could also have been decided to reduce the size of the storage organs. To increase the size of a storage organ according to the invention it is decided to over-express the gene encoding the potassium channel previously described. In fact, the inventors discovered that by over-expressing the VvSOR gene in the grape berry cell of Vitis vinifera , whilst transferring at least one additional copy of the gene of interest into the cell by transgenesis, the size of the berry was 1.7 times greater than the reference size of a “wild” grape berry. The reference size of a “wild” grape berry is understood to mean an average size calculated from the size of all the grape berries contained on one representative sample taken from a variety of corresponding “wild” Vitis Vinifera that has not been subjected to any genetic transformation according to the invention.

In the same experiments the inventors discovered that the over-expression of the gene VSOR causes a major increase of up to 20% in the quantity of tartaric acid produced an accumulated in the berries.

For this invention the inventors increased the expression of the gene of interest involved in the growth of the storage organs. To this they cloned a complementary VvSOR (or DNAc VvSOR) or SEQ ID No. 1 DNA deriving from the vine Vitis vinifera in a CAMV35S vector to obtain a recombinant vector. Embryogenic cells of the vine Vitis were then transformed with this recombinant vector to insert in the genome of these cells at least one additional copy of the gene. The cell to be transformed may be selected from the cells of a plant capable of reproducing an entire organism. This method has been described in more detail in the document Torregrosa, L. (1998) Vitis 37, 91-92.

The gene encoding a polypeptidic sequence having at least a 40% similarity with the polypeptidic sequence deduced from the sequence of the gene SEQ ID No. 1 is then used to increase the expression of the corresponding protein in the cells of each plant, the size and/or organic acid composition of whose storage organ is required to be modified.

According to the invention a cell transformed by the method described above is then obtained.

Also according to the invention a transformed plant is obtained by the method and by the transformed cell described above.

The invention also provides for a marker of a gene comprised in a phenotype relating to a size or organic acid composition of a storage organ. The marker comprises a nucleotidic sequence encoding a polypeptidic sequence having at least a 40% similarity with a polypeptidic sequence deduced from all or part of the nucleic sequence SEQ ID No. 1, or all or part of a nucleotidic sequence complementary to SEQ ID No. 1, or of a nucleotidic sequence SEQ ID No. 2 or SEQ ID No. 3. This percentage similarity takes into account the differences that exist from one plant species to another, whilst guaranteeing that the gene encodes a potassium channel involved in the size of organic acid composition phenotype of the storage organ, or having a behaviour similar to the potassium channel resulting from the expression of the SEQ ID No. 1 gene.

This marker may be used to detect plants in which a gene is expressed encoding a polypeptidic sequence having at least a 40% similarity with a polypeptidic sequence deduced from the nucleic sequence SEQ ID No. 1. These plants, for which the gene similar to the gene of the SEQ ID No.1 has been identified, may then be modified according to the invention by means of this detected gene.

This marker may be detected by molecular hybridisation between the nucleotidic sequence of a marker of a given plant and all or part of the sequence SEQ ID 1, the sequence complementary to SEQ ID No.1 or the sequence SEQ ID No. 2 or SEQ ID No. 3.

The nucleotidic sequence of the marker may be in the form of a DNAc, a non-encoding DNA strand or an RNAm. The nucleotidic sequence of the marker may be in any form likely to be detected by molecular hybridisation with all or part of the sequence SEQ ID No.1, the sequence complementary to SEQ ID No. 1, of SEQ ID No. 2 or the sequence SEQ ID No. 3.

The nucleotidic sequences SEQ ID No. 2 and SEQ ID No. 3 form a front primer and a rear primer of the SEQ ID No. 1. These primers enable the presence of the SEQ ID No. 1 to be detected by hybridisation of the DNAc, the non-encoding DNA or the RNAm deriving from the gene SEQ ID No.1 These primers correspond to nucleotidic sequences flanking the gene of the SEQ ID No. 1 sequence.

The invention also relates to a nucleotidic primer which comprises a nucleotidic sequence encoding a polypeptidic sequence having at least a 40% similarity with a polypeptidic sequence deduced from all or part of the nucleotidic sequences SEQ ID No. 2 or SEQ ID No. 3. These sequences SEQ ID No. 2 and SEQ ID No. 3 correspond to a front flanking sequence and a rear flanking sequence of SEQ ID No. 1 respectively. These primers may serve to achieve a RNA-PCR, a method well known to the person skilled in the art. This method enables the gene of interest to be amplified then used to modify the expression of a gene encoding a potassium channel of a plant according to the invention.

To select plants or cells of plants relative to a “size” phenotype or the organic acid composition of the storage organs of the said plants, the invention provides that the expression is measured of a gene encoding a outward rectifier potassium channel in the cells of the storage organs or the tissues supplying that organ.

The invention provides that the gene whose expression is measured encodes a polypeptidic sequence having at least a 40% similarity with a polypeptidic sequence deduced from the gene defined by the sequence SEQ ID No. 1

In order to measure the expression of the gene of interest either a measurement of a quantity of RNAm deriving from a transcription of the gene encoding the potassium channel, or a measurement of a quantity of proteins resulting from the expression of this gene is carried out according to the invention. The measurement of these quantities may be carried out by molecular biology methods well known to the person skilled in the art. For example, the measurement of these quantities may be carried out by a molecular hybridisation method (Northern Blot), a quantitative reverse-PCR method or by a Western Blot method.

The inventors discovered that the expression of the VvSOR gene corresponding to SEQ ID No. 1 is particularly observable in the cells or aerial parts of the plant Vitis vinifera and in the storage organs of this same plant.

The inventors discovered that the VvSOR gene encodes a protein comprising 796 amino acids, with a molecular weight of approximately 91.2 kDa. The protein comprises a typical structure of the Shaker channels of the plants (see FIG. 1). This protein comprises an N-terminal region, a hydrophobic core consisting of 6 transmembranous segments (called S1 to S6) and a pore forming a domain (P) between S5 and S6, a long C-terminal region containing a site assumed to be a cyclic nucleotide link site, followed by an ankyrin domain consisting of 6 repeated motifs, followed by a K_{HA} domain (Zimmermann, S. and Sentenac, H. (1999) Current Opinion in Plant Biology 2, 477-482), rich in hydrophobic amino acids and acids. All the terminal regions C and N of the plant channels of the Shaker type are assumed to have a cytoplasmic location.

The development of the grape berries is characterised by a first growth phase, an intermediate stagnation phase followed by a transition called veraison, then a second growth phase. In particular, the inventors discovered that the quantity of RNAm resulting from the expression of the gene SEQ ID No. 1 was extremely important in the course of the development of the berry, and that the quantity of proteins was very high at the time of development and after the development of the berry.

The measurements of expression of the gene of interest may therefore preferably be carried out at particular times of development of the plant. In a particular exemplary embodiment of the invention the moment of development of the storage organ is preferred for sampling the cells and measuring their RNAm rate or the rate of proteins, expressed by the gene of interest in a transformed cell.

By applying the selection process to cells of a storage organ at a particular time of development, it is advantageously possible to identify the plants having large size storage organs specifically because of the over-expression of the gene, and not due to exposure to sunshine or particularly favourable feeding. Because of this the plants thus identified have a good chance of stably and reproducibly producing new generations of plants that also have this genetic characteristic.

The selection of non-transformed plants, which would for example have a powerful transcription factor compared with the SOR gene present in the reference plants, is therefore avoided.

It is also possible to select the transformed plants by means of an antibiotic. In fact, during the transformation of cells of storage organs provision can be made for the recombinant vector to include a gene resistant to an antibiotic in addition to the gene of interest.

Moreover, this method may be method of verification and may be used to check that the plants have been correctly transformed.

Prior to the stage of measuring the expression of the gene, plants may be selected whose storage organs have a size at least 25% greater by weight than a reference size of storage organs.

Reference size of storage organs is understood to refer to an average size calculated from a measurement of all the storage organs deriving from a sample of wild storage organs, i.e. organs which have not been transformed and which are representative of the species and variety, line or cultivar considered. This gives a reference size for each variety, line or cultivar of any plant species considered.

For example, the size of storage organ may be quantified by measuring the weight of 100 storage organs for a given wild species. This gives a reference weight of storage organs for a given non-transformed plant. In parallel with, the weight of 100 other storage organs of a plant that has been modified according to the invention is measured. These two weights are then compared to deduce from it any transformation of the plant. In one example the inventors discovered that the weight of a storage organ derived from a transformed plant according to the invention was 1.7 times greater than the weight of a storage organ derived from a non-transformed plant.

The invention also relates to the use of a gene encoding a outward rectifier potassium channel for modifying a phenotype relating to a size or the organic acid composition of the storage organs of a plant. The gene encodes a polypeptidic sequence having at least a 40% similarity with a polypeptidic sequence deduced from the nucleotidic sequence SEQ ID No. 1. This gene is used in the invention by modifying its expression. In particular, in a preferred embodiment of the invention, it has been decided to over-express the gene in cells of the storage organ in order to increase the size of the storage organs. But it might also be decided to inhibit its expression in order to reduce the size of the storage organs. This gene enables the plants to be derived that have a gene whose expression enables a potassium channel to be obtained having physiological properties similar to the VvSOR potassium channel

The invention also relates to an antibody directed against all or part of a polypeptidic sequence resulting from the expression of a gene, which polypeptidic sequence has at least a 40% similarity with a polypeptidic sequence deduced from the nucleotidic sequence SEQ ID No. 1. Antibodies, within the meaning of this invention, refer to polyclonal or monoclonal antibodies or fragments (e.g. the fragments F(ab)′2, F(ab) or even fragments comprising a domain of the initial antibody recognising the polypeptide or the target polypeptide fragment according to the invention.

The antibody may be obtained by the method of hybridomes well know to the person skilled in the art. This method of hybridomes would in this case consist in obtaining a fusion between cells producing antibodies directed against a polypeptidic sequence derived from all or part of the SEQ ID No. 1 with a myelomatous cell.

The gene for which one seeks to detect the presence of the corresponding polypeptide encodes a polypeptidic sequence having at least a 40% similarity with a polypeptidic sequence deduced from all or part of the nucleotidic sequence SEQ ID No. 1.

Finally, the invention relates to a kit for detecting all or part of the polypeptide previously described in a sample containing a mixture of polypeptides, characterised in that comprises
an antibody, previously described, the antibody being detectable by methods of marking the antibody well known to the person skilled in the art.

Figures

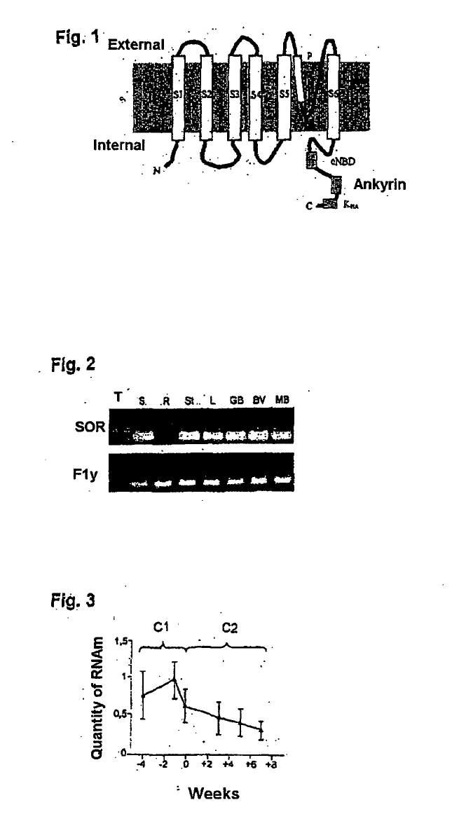

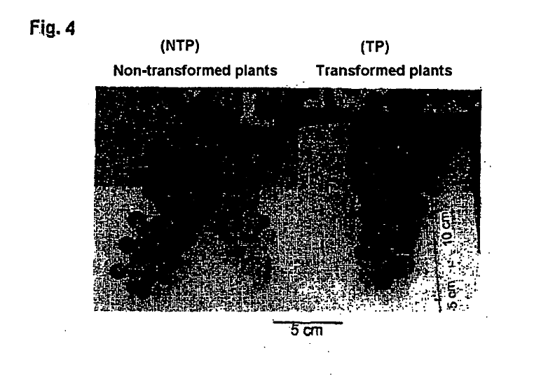

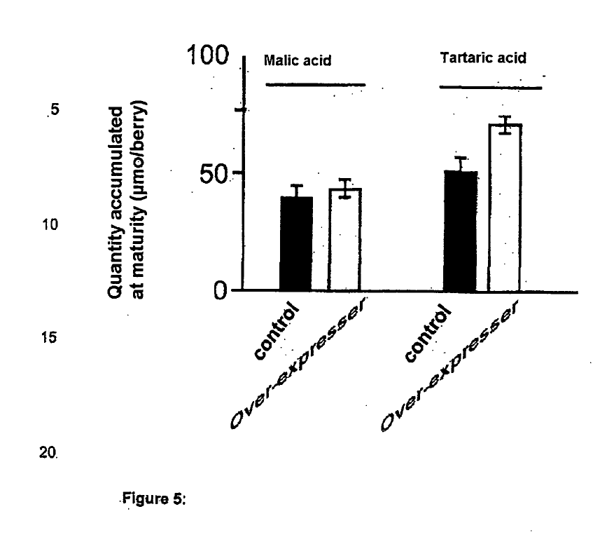

FIG. 1: A diagrammatic representation of a potassium channel encoded by the VvSOR gene of Vitis vinifera;
FIG. 2: Gel of an electrophoretic migration of products of amplification by quantitative RT-PRC of the RNAm produced by the gene VvSOR in cells or tissues sampled at different points on a transformed plant (TP) or a non-transformed plant (NTP);
FIG. 3: A graphic representation of a quantity of RNAm VvSOR produced by grape berries of Vitis vinifera in the course of the growth of such berries. The time reference is the “veraison” stage, which separates the two growth phases C1 and C2.
FIG. 4: A first photograph representing bunches of grapes in the veraison stage, derived from transformed plants; and
FIG. 5: A graphic representation of the accumulated quantities of tartaric and malic acid, on the one hand in the berries harvested at maturity from non-transformed plants (control), and on the other hand in berries harvested at maturity from transformed plants over-expressing the VvSOR gene (over-expresser).

### EXAMPLES

Example of Implementation of the Method for Obtaining Transformed Cells and Verification

To obtain a transformed plant an expression of the gene VvSOR (or SEQ ID No. 1) is modified, this consisting in carrying out a transgenesis. To carry out this transgenesis an embryogenic cell is transformed with a recombinant vector which incorporates an additional copy of the cellular gene of interest. It is then checked that the transgenesis has been correctly carried out by measuring the expression of the cellular gene and the foreign gene.

Equipment and Methods

The plant as well as the methods used for carrying out the genomic analyses were described by Pratelli, R., et al., 2002, Plant Physiology 128, 564-577.

Screening of a Bank of DNAc of Vitis Vinifera

Bank of DNAc of berries of Vitis Vinifera is screened to identify the SEQ ID No. 1 with two probes prepared from DNAc of Arabidopsis thaliana (DNAc AtSKOR and DNAc AtKCO1), each of these DNAc's encoding a rectifying outward rectifier potassium channel of the Shaker type. The screening was carried out according to a method well known to the person skilled in the art (Fillion, L. Ageorges, A., Picaud, S., Coutos-Thévenot, P., Lemoine, R., Romieu, C. and Delrot, S. (1999) Plant Physiology 120, 1083-1093; Gaymard, F. et al. (1998)( Cell 94, 647-655; Czempinski, K., Zimmermann, S., Ehrhardt, T. and Müller-Röber, B. (1997) The EMBO Journal 16, 2565-2775; Church, G. W. and Gilbert, W. (1984) Proceedings of the National Academy of Sciences of the United States of America 81, 1991-1995).

Transformation of the Berry

In order to transform the vine Vitis Vinifera a transgenesis is carried out in order to increase the expression of the gene obtained after screening the DNAc bank of cells of berries of Vitis vinifera. Embryogenic cells of Vitis vinifera are prepared for the purpose of transforming them with DNAc (or SEQ ID No. 1) encoding a potassium channel of Vitis vinifera, and obtained after screening the DNAc just described.

This transformation is specifically aimed at transferring at least one additional copy of the DNAc or SEQ ID No. 1 in the cytoplasm of the cell, and integrating it in the nuclear DNA of the host cell. In parallel with a vector is prepared as described by Torregrosa, L. (1998, Vitis 37, 91-92) in order to obtain a future recombinant vector including the nucleotidic sequence isolated by screening the bank.

Amplification of the DNAC VvSOR

In order to amplify the DNAc an RNA-PCR and a semi-quantitative RNA-PCR are carried out (Pratelli, R., Lacombe, B., Torregrosa, L., Gaymard, F., Romieu, C., Thibaud, J. B. and Sentenac, H. (2002) Plant Physiology 128, 564-57). The specific primers for VvSOR are the front primer SOR or SEQ ID No. 2 and the rear primer SOR or SEQ ID No. 3. This RNA-PCR is carried out from RNAm's derived from cells of a transformed plant (TP) and from cells derived from a non-transformed plant (NTP). The RNA's were sampled from different points on the transformed plant (TP) and at different points on the non-transformed plant (NTP), FIG. 2. In particular, for each of the plants TP and NTP, RNA's were sampled from tail cells of the fruit (S), root cells (R), cells of the stem (St), leaf cells (L), cells of green berries (GB), cells from berries at the time of veraison (BV) and cells of mature berries (MB).

Quantifications

The RNAm's and the proteins of the samples taken from transformed and non-transformed plants were isolated. Using molecular hybridisation methods (Northern Blot), quantitative Reverse-PCR and Western Blot), the quantity of RNAm's and proteins derived from the expression of the gene SEQ ID No. 1 was determined.

Electrophysiological Study of the Channel Coded by the Gene Isolated after Screening of the DNAc Bank of Grape Berries of Vitis vinifera in a Xenope Ovocyte.

The DNAc which was isolated after screening of the bank is cloned in a vector, and such a recombinant vector is then injected into a Xenope ovocyte according to a “patch clamp” method, a method well known to the person skilled in the art. This “patch clamp” method enables the electrophysiological properties of transmembranous channels vis-à-vis certain ions to be studied.

### RESULTS

Result of Screening of the DNAc bank, FIG. 1

An insert of 2.4 kbp was detected and sequenced. This insert served to isolate the complete gene of Vitis Vinifera, which encodes a new outward rectifier potassium channel similar to the potassium channels of Arabidopsis, of the Shaker type. A length of this complete DNAc is 2.5 kbp. This complete DNAc is called VvSOR (Vitis vinifera Shaker-like Outward Rectifier).

The inventors discovered that the VvSOR gene encodes a protein comprising 796 amino acids with a molecular weight of approximately 9.1 kDa. The protein has a typical structure of the Shaker channels of the plants (see FIG. 1). This protein comprises an N-terminal region, a hydrophobic core consisting of 6 transmembranous segments (called S1 to S6) and a pore forming a domain (P) between S5 and S6, a long C-terminal region containing a site assumed to be a cyclic nucleotide link site, followed by an ankyrin domain consisting of 6 repeated motifs, followed by a K_{HA} domain (Zimmermann, S. and Sentenac, H. (1999) Current Opinion in Plant Biology 2, 477-482), rich in hydrophobic amino acids and acids. All the terminal regions C and N of the plant channels of the Shaker type are assumed to have a cytoplasmic location.

The VvSOR gene has 84% identity vis-à-vis the AtSKOR gene (Gaymard, F. et al. (1998) Cell 94, 647-655) and 79% vis-à-vis the AtGORK gene (Ache, P. et al. ( 93-98). A comparison of the structure of the genes and the position of the introns of VvSOR, AtSKOR and AtGORK showed that the gene of the Vine Vitis was closer to the AtGORK gene, mainly because these two genes have specific intron positions which are not found in the AtSKOR gene (Pilot, G., et al., 2003, Journal of Molecular evolution 56, 418-434). The SOR gene is therefore the horthologue of the GORK gene in the vine Vitis vinifera.

Results of the Amplification, Location of the Expression of the VvSOR Gene, FIGS. 2 and 3

The RNAm's derived from the expression of the SEQ ID No. 1 gene are present in the tail cells of the fruit (S), in the cells of green berries (GB), in the cells of berries at the time of veraison (BV) and in the cells of mature berries (MB). But the RNA's derived from the expression of the gene SEQ ID No. 1 are not present in the roots®, FIG. 2. The inventors were therefore able to show that the expression of the gene was observable in the aerial parts of the plant, particularly in the leaves, the young shoots and the berries, but not in the roots.

The RNA's derived from the transformed plant are present in a relatively larger quantity that the RNA's derived from the non-transformed plant (FIG. 2).

The inventors also estimated the quantity of RNAm produced during the growth of the grape berries of the plant, FIG. 3. FIG. 3 represents a quantity of RNAm produced during the development of the grape berry as a function of time. In particular, the development of the grape berries is characterised by a first growth stage C1, a stagnation stage followed by a transition stage called veraison, then a second growth stage C2. The inventors observed that the quantity of RNAm is at its maximum at the time of veraison, indicating that the detection of the transformed plants should preferably be carried out at the time of veraison.

Results of the Transformation, FIGS. 2, 4 and 5

After electrophoretic migration of the amplified SEQ ID No.1 DNAc's encoding the potassium channel of Vitis Vinifera on the electrophoresis gel, the presence of the DNAc is only observed in the aerial parts and in the berries for the transferred and non-transformed plants (FIG. 2). On the other hand, the DNAc is not present in the roots. The implementation of the method of selection should preferably be carried out on all the cells in which the gene of interest has been detected.

FIG. 4 shows a first photograph of bunches of grape berries deriving from non-transformed plants, and a second photograph of grape berries from transformed plants. The inventors measured the weight of numerous representative samples consisting of 100 berries deriving from transformed plants, and 100 berries deriving from non-transformed plants. A comparison of the non-transformed and transformed plants was carried out in two different conditions.

The first of these conditions is a fruit-bearing cycle obtained under climatised and illuminated glass, completed by harvesting at maturity in spring.

The second of these conditions is a fruit-bearing cycle obtained under non-climatised and non-illuminated glass, completed by harvesting at maturity in the summer period.

In the two conditions described above the inventors observed with the naked eye that the size of the berries derived from transformed plants was greater than the size of the berries derived from non-transformed plants. In the first of the experimental conditions described above, the inventors observed that the size of the berries, estimated by the average weight of 100 berries derived from transformed plants is 1.3 times larger than the size of the berries derived from non-transformed plants. In the second of these experimental conditions described above the inventors observed that the size of the berries, estimated on the basis of the average weight of 100 berries derived from transformed plants is 1.7 times greater than the size of the berries derived from non-transformed plants.

The inventors observed that the quantity of malic acid accumulated in the ripe berries is significantly different in the transformed plants from that in the non-transformed plants (FIG. 5). Conversely, the inventors observed that the quantity of tartaric acid accumulated in the ripe berries is far greater (at least 20%) in the transformed plants than in the non-transformed plants (FIG. 5).

Result of the Electrophysiological Study in Xenope Ovocyte

The inventors discovered that the protein coded by VvSOR was a potassium channel allowing a flux of potassium ions from the cell. This channel is preferably permeable to the potassium ion, and is blocked by inhibitors similar to those used to block the potassium channels coded by the genes AtSKRO and AtGORK of the model plant Arabidopsis thaliana.

The potassium channel coded by the VVSOR gene is dependent on the external and internal potassium concentration. A reduction in the external potassium concentration involves a reduction in the outward potassium flow. This channel is also dependent on the pH inside and outside the cell. In fact, a reduction in external or internal pH involves a considerable reduction in the outward potassium flow.

Example of the implementation of the method of selecting the plant: molecular detection by measurement of the quantity of proteins produced or by the quantity of RNAm produced.

After transformation of the cells of grape berries, the cells of grape berries are crushed to extract the RNAm's and proteins at particularly favourable times for detecting an optimum quantity. The quantity of RNAm VvSOR produced by the gene of Vitis vinifera VvSOR is determined by quantitative Reverse-PCR or molecular hybridisation (Northern blot), and the quantity of proteins deriving from the translation of this RNAm is determined by immunodetection (Western blot method). Each of these quantities is compared to a quantity of reference RNAm and a quantity of reference proteins corresponding to a quantity of RNAm and proteins normally present in the cells of non-transformed plants respectively.

A time is chosen when there is the most chance of finding in large quantities the expression of the gene to be measured. In particular, a time in the first growth phase C1 of the berry, FIG. 3, is selected for measuring the quantity of RNAm, and a time in one of the growth phases C1 or C2, preferably C2, or a time after development of the berry, is selected for measuring the quantity of protein.

### BIBLIOGRAPHICAL REFERENCES

Dutruc-Rosset, G. (2003) in: Bull. O. I. V., special issue, pp. 94 O. I. V., Paris.

Hrazdina, G., Parsons. G. F. and Mattick, L. R. (1994) American Journal of Enology and Viticulture 35, 220-227.

Hale, C. R. (1977) Vitis 16, 9-19.

Very, A.-A. and Sentenac. H. (2003) Annual Review of Plant Biology 54, 575-603.

Pratelli, R., Lacombe, B., Torregrosa, L., Gaymard, F., Romieu, C., Thibaud, J. B. and Sentenac, H. (2002) Plant Physiology 128, 564-577.

Fillion, L., Ageorges, A., Picaud, S., Coutos-Thévenot, P., Lemoine, R., Romieu, C. and Delrot, S. (1999) Plant Physiology 120, 1083-1093.

Sambrook, J., Fritsch, E. F. and Maniatis, T. (1989), Cold Spring Harbor.

Gaymard, F. et al. (1998) Cell 94, 647-655.

Czempinski, K., Zimmermann, S., Ehrhardt, T. and Müller-Röbber, B. (1997) The EMBO Journal 16, 2565-2775.

Church, G. W. and Gilbert, W. (1984) Proceedings of the National Academy of Sciences of the United States of Anerica 81, 1991-1995.

Lacombe, B. and Thibaud, J.-B. (1998) Journal of Membrane Biology 166, 91-100.

Lacombe. B. Pilot, G., Gaymard, F., Sentenac, H. and Thibaud. J.-B. (2000) FEBS Letters 466, 351-354.

Lacombe, B., Pilot, G., Michard, E., Gaymard, F., Sentenac, H. and Thibaud, J.-B. (2000) The Plant Cell 12, 837-851.

Zimmermann, S. and Sentenac, H. (1999) Current Opinion in Plant Biology 2, 477-482.

Langer, K. et al. (2002) The Plant Journal 32, 997-1009.

Ache, P., Becker, D., Ivashikina, N., Dietrich, P., Roelfsna, M. R. and Hedrich, R. (2000) FEBS Letters 486, 93-98.

Pilot, G., Pratelli, R., Gaymard, F., Meyer. Y. and Sentenac, H. (2003) Journal of Molecular Evolution 56, 418-434

Decroocq, V., Fave, M. G., Hagen. L., Bordenave, L and Decroocq, S. (2003) Theoretical and Applied Genetics 106, 912-22.

Kanellis, A. K and Roubelakis-Angelakis, K. A (1993) in: Biochemistry of Fruit Ripening, pp. 189-234 (Seymour, G., Taylor, J. and Tucker. G., Eds.) Chapman Hall., London.

Winkler, A., Cook, J., Lider, J. A. and Kliewer, W. M. (1974) University of California press, Berkeley.

Blatt. M. R. and Gradmann, D. (1997) Journal of Membrane Biology 158,241-256.

Roberts, S. K. and Tester, M. (1995) The Plant Journal 8, 811-825.

de Boer, A. H. and Wegner, L. H. (1997) Journal of Experimental Botany 48, 441-449.

Madeja, M. (2000) News in Physiological Sciences 15. 15-19.

Geiger, D., Becker, D., Lacombe, B. and Hedrich. R. (2002) The Plant Cell 14. 1859-68.

Mouline, K. et al. (2002) Genes and Development 16, 339-350.

Hosy, E. et al. (2003) Proceedings of the National Academy of Sciences of the United States of America 100, 5549-54.

Blanke, M. M., Pring, R. J. and Baker, E. A. (1999) Journal of Plant Physiology 154, 477-481.

Possner, D. R. E. and Kliewer, W. M. (1985) Vitis 24, 229-240.

Coombe. B. G. (1987) American Journal of Enology and Viticulture 38, 120-127.

Torregrosa, L. (1998) Vitis 37, 91-92.

Becker, D., Hoth, S., Ache, P., Wenkel, S., Roelfsema, M. R. G., Meyerhoff, O., Hartung, W. and Hedrich, R. (2003) FEBS Lett (in press).

Baizabal-Aguirre, V. M. Clemens, S., Uozomi, N. and Schroeder, J. I. (1999) Journal of Membrane Biology 167, 119-125.

1. A method for obtaining a transformed plant having a modified phenotype relating to a size or the organic acid composition of a storage organ of the plant, comprising the step of:
modifying the expression of a gene encoding an outward potassium channel in one or more cells of the plant wherein the cells are selected from the group consisting of the cells of the storage organ and the cells in the tissues supplying the storage organ.

2. The method according to claim 1, further comprising the steps of:
transforming at least one cell of the plant with the gene encoding the outward potassium channel,
selecting the at least one transformed cell, and regenerating a transformed plant from the selected transformed cell.

3. The method according to claim 1, wherein the gene whose expression is modified encodes a polypeptide sequence having at least a 40% similarity with a polypeptide sequence deduced from the nucleotide sequence encoding an outward potassium channel derived from Vitis Vinifera (VvSOR).

4. The method according to claim 1, wherein the gene is over-expressed.

5. (canceled)

6. A transformed plant, obtained by the method according to claim 2.

7. A method of selection of a plant having a modified phenotype relating to a size of the storage organs of the said plant and/or organic acid composition, wherein the expression of a gene encoding an outward potassium channel of the plant in the cells of the storage organs or in the tissues supplying the storage organs is measured.

8. The method according to claim 7, wherein the gene whose expression is measured encodes a polypeptide sequence having at least a 40% similarity with a polypeptide sequence deduced from the nucleotide sequence encoding an outward potassium channel derived from Vitis Vinifera (VvSOR).

9. The method according to claim 8, wherein a quantity of mRNA derived from a transcription of the gene is measured, or a quantity of proteins resulting from the expression of the gene is measured.

10. The method according to claim 9, wherein the measurement of the quantity of mRNA is carried out during the development of the storage organs, and in that the measurement of the proteins is carried out during or after the development of the storage organs.

11. A cell of a plant, wherein the cell over-expresses a gene encoding an outward rectifier potassium passage whose polypeptide sequence has at least a 40% similarity with a polypeptide sequence deduced from the nucleotide sequence encoding an outward rectifier channel derived from Vitis Vinifera (VvSOR).

12. A plant, wherein the plant over-expresses a gene encoding an outward rectifier potassium channel of the said plant whose polypeptide sequence has at least a 40% similarity with a polypeptide sequence deduced from the sequence encoding an outward rectifier channel derived from Vitis Vinifera (VvSOR).

13. (canceled)

14. The use according to claim 13, wherein the gene encodes a polypeptide sequence having at least a 40% similarity with a polypeptide sequence deduced from the sequence encoding an outward rectifier potassium channel derived from Vitis Vinifera (VvSOR).

15. The use according to claim 13, wherein the gene in the cells of the storage organ is over-expressed.

16. An antibody, wherein the antibody is directed against all or part of a polypeptide derived from the expression of a gene encoding an outward rectifier potassium channel of a plant.

17. The antibody according to claim 16, wherein the gene encodes a polypeptide sequence having at least a 40% similarity with a polypeptide sequence deduced from the sequence encoding an outward rectifier potassium channel derived from Vitis Vinifera (VvSOR).

18. A method for detecting the presence of all or part of a polypeptide resulting from the expression of a gene encoding an outward rectifier potassium channel of a plant in a sample comprising a mixture of polypeptides, wherein it comprises the following stages:
putting the sample in contact with an antibody according to claim 16, and
detecting an antigen/antibody complex formed.

19. The method according to claim 18, wherein the gene encodes a polypeptide sequence having at least a 40% similarity with a polypeptide sequence deduced from the sequence encoding an outward rectifier potassium channel derived from Vitis Vinifera).

20. A kit for detecting all or part of a polypeptide produced from a gene encoding a potassium channel of a plant in a sample containing a mixture of polypeptides, wherein it comprises an antibody according to claim 16.

21. The detection kit according to claim 20, wherein the gene encodes a polypeptide sequence having at least a 40% similarity with a polypeptide sequence deduced from the sequence encoding an outward rectifier potassium channel derived from Vitis Vinifera (VvSOR).


SEQUENCE LISTING (3 sequences)

SEQ ID NO: 1 - DNA, 2370 bp, Vitis vinifera
atgtcgttttcttgtgcaaaagccttcttccaacggttttgtgttgaagagttccaaatggagagaactagcctaggttccgtcttcttagtcatctcctcccatctcttggggccagaatcaaccaggcaacaaagcttcaaaaacacataatttctcctttcagtccccgttacagggcttgggaaatgttgctgattattctggtcatctactctgcctgatctgcccatttgagtttggatttctgccctacaagcaagacgcgcttttcatcttcgataacattgtcaatggcttcttcgccatcgatatcgttctcactttctttgtagcatacctcgacacagaaacttatcttcttgttgatgatgccaagaaaattgcaatcaggtacatatctacctggtttatcttcgatgtctgttcgacagcgccatttgaagctttcagcctcctgttcacaaagcataacagtggactcggctataaagcactcaacatgctcaggctctggcgactgagacgagtcagctccctgtttgcaagactagagaaggacatccggtttaactacttctggattcgatgcataaaactcacttctgtaactctgtttgcagtacactgtgctggatgctttaactatctgattgcagatagatacccggatccagaacgaacctggattggtgcagtctatccaaatttcaaagaagagaacctctgggacagatatgtaacttcaatttactggtctattactacactaactactactggttatggagacttgcatgctgagaacccaagagagatgctgtttgatattttttacatgctattcaacttgggattaacatcttacctcattgggaacatgaccaatcttgttgttcactggaccagccggaccagagattttagggatacagtcaggtctgcttcagagtttgcaacaaggaatcaattgcccccacgcattcaggtcagatgctgtcgcacttatgtctcaagttcaaaacagaaggattgaaacaacaagacactttgaatggcctgccaagagccattcgctccagcattgcacactacctcttcttccctatcgctcaaaatgtctatcttttccagggtgtttctcaggacttccttttccaactggtttctgaagtggaggctgagtatttcccacctagagaagatgtgattctacagaaggaggcttcaacagatatatatattcttgtctcgggagcggtggatttgatagcatatattgacggacatgatcagattctcggaaagctgttgcaggggatgtgtttggagagattggggttttatgttataggccacaatcgttaacagtccggacctctgagctttctcagatactaagattaagcagaacttcactgatgaacgcaatccaagcaaatatggaagatggaccaattattatgaaccatcttttcaagaaactgaaagggctagaaagctcaggctttacagacccacatatggacccagattccatcctcagagaatggattgatggagtaccaccaggaggaagcctttcccatgctggatgtcatgatcaatcaccacatggagatccatcaatacaagaagcaagggacataggtttactgggatcagaagctacaaagaagagtaaagcagacaaagctcatgagtcgactggatgcgggatcgatgcaaattcagcagctgaggatggccaaacggctcttcatgttgctgtctgcaacgggcatcttgaaatggttagaattctgctagaaagaggagcaaatgtgaacaaaaaggatgctagagggtggaccccaaaagctttagcggaacaagaaggaaaaaaaagcatatatgacctcttactaagttataaaatagaaggttattagatgaacacaaaattcattttattgggtcagacgcagctgactgttgtactagtcaaggtctacatacaagaacgggggggcccaattttcacaactctcaatttaaaaaggtatccacaaattccaattcaggcagtcctagccctccggcaacaaagatgttatgacattgaccaagaggagagtcactatccacagacagtttcaaaatgcaagtacatcacaggggcagcttgggaagttgattattctacctgattcaatagaagagctactgcaaattgctggtcaaaagtttggaggctacaatcccaccaaagtcgttagtgcagggaatgcagaaatagatgacataagcgttatccgagatggggatcatctgtttctacttcaaaatgagaatggaactacaattacaatgttacctaatggttactga

SEQ ID NO: 2 - RNA, 22 nt, Vitis vinifera
gcaaaggcaggaaagccggggc

SEQ ID NO: 3 - RNA, 19 nt, Vitis vinifera
cacaaacaaaaagccgacc